SEQUENCE LISTING (8 sequences)

SEQ ID NO: 1 - DNA, 1454 bp, synthetic construct
  source 1..1454: mol_type = other DNA
cagtgagggtaacaaagtcaatttttttaacattgggtatatgtatatatttgagggaattgtcagtgagaagaatacatggtaggaaatgacacacgtcccaagcaacagatggatggttgagaatgaacccggtcattgaattgacattagaaaatgttctaaaatatatttttatcgacgttactgatcagaaacaaagtcctccgccagccagaggcatatcctaatgattgtacctttcctcactgacaattcccattaattagctgctgccactgcatagcggctataatataatggtagagggcccatggagctttccttttcagtgaacatgggttagtcgacaaaagaaaatgttcaagttgaaagaggagcggtggccctcaaaggttcccgtcacttggttgctttttgtctaaggaaacagtggtatcagaatccaaatcttctacgtaatgtcccattcagtgagaagaatacatggtaggaaatgacacacgtcccaagcaacagatggatggttgagaatgaacccggtcattgaattgacattagaaaatgttctaaaatatatttttatcgacgttactgatcagaaacaaagtcctccgccagccagaggcatatcctaatgattgtacctttcctcactgacaattcccattaattagctgctgccactgcatagcggctataatataatggtagagggcccatggagctttccttttcagtgaacatgggttagtcgacaaaagaaaatgttcaagttgaaagaggagcggtggccctcaaaggttcccgtcacttggttgctttttgtctaaggaaacagtggtatcagaatccaaatcttctacgtaatgtcccattcagtgagaagaatacatggtaggaaatgacacacgtcccaagcaacagatggatggttgagaatgaacccggtcattgaattgacattagaaaatgttctaaaatatatttttatcgacgttactgatcagaaacaaagtcctccgccagccagaggcatatcctaatgattgtacctttcctcactgacaattcccattaattagctgctgccactgcatagcggctataatataatggtagagggcccatggagctttccttttcagtgaacatgggttagtcgacaaaagaaaatgttcaagttgaaagaggagcggtggccctcaaaggttcccgtcacttggttgctttttgtctaaggaaacagtggtatcagaatccaaatcttctacgtaatgtcccattcatcctaccaatgtccatatgaattcctctggacgttaaaaaatggttgcacgtggttgtcttcaggatcacaccagtttatacatttggaccacaaaatagagattgttcatcaaggatactagtcagcaattaattcctaaatatctcttatgacacacaccctt

SEQ ID NO: 2 - DNA, 408 bp, synthetic construct
  source 1..408: mol_type = other DNA
cagtgagaagaatacatggtaggaaatgacacacgtcccaagcaacagatggatggttgagaatgaacccggtcattgaattgacattagaaaatgttctaaaatatatttttatcgacgttactgatcagaaacaaagtcctccgccagccagaggcatatcctaatgattgtacctttcctcactgacaattcccattaattagctgctgccactgcatagcggctataatataatggtagagggcccatggagctttccttttcagtgaacatgggttagtcgacaaaagaaaatgttcaagttgaaagaggagcggtggccctcaaaggttcccgtcacttggttgctttttgtctaaggaaacagtggtatcagaatccaaatcttctacgtaatgtcccatt

SEQ ID NO: 3 - DNA, 284 bp, synthetic construct
  source 1..284: mol_type = other DNA
ttcaggatagggaggtctgaactcagttgaaggaatagtaaaagaagacaaagcaactattagagcttaagaagagtcccattttagtattagtgacttcatactacaaagaaaacttagggaaaaaaaaaactcaattagaaaaaagtctttttgacaagattaggattactgatatgggtcttagcatgtaagcaatcatgttcatgcattccaattctttctattcttgttttccttttttcctttttttcacctaatatttgttgagatgaaaagtggct

SEQ ID NO: 4 - DNA, 70 bp, synthetic construct
  source 1..70: mol_type = other DNA
ggaaaaaaaaaactcaattagaaaaaagtctttttgacaagattaggattactgatatgggtcttagcat

SEQ ID NO: 5 - DNA, 21 bp, synthetic construct
  source 1..21: mol_type = other DNA
ttcaggatagggaggtctgaa

SEQ ID NO: 6 - DNA, 22 bp, synthetic construct
  source 1..22: mol_type = other DNA
agccacttttcatctcaacaaa

SEQ ID NO: 7 - DNA, 20 bp, synthetic construct
  source 1..20: mol_type = other DNA
cagtgagggtaacaaagtca

SEQ ID NO: 8 - DNA, 20 bp, synthetic construct
  source 1..20: mol_type = other DNA
aagggtgtgtgtcataagag

(19) United States
(12) Patent Application Publication
(10) Pub. No.: US 2026/0114395 A1
(43) Pub. Date: Apr. 30, 2026

(54) METHOD FOR BREEDING NEW GRAPE VARIETY WITH RED FLESH AND SEEDLESSNESS BASED ON MOLECULAR MARKERS

(71) Applicant: INSTITUTE OF BOTANY, THE CHINESE ACADEMY OF SCIENCES, Beijing (CN)

(72) Inventors: Zhenchang LIANG, Beijing (CN); Yi WANG, Beijing (CN); Peige FAN, Beijing (CN); Kangyi DING, Beijing (CN); Chong REN, Beijing (CN); Yangfu KUANG, Beijing (CN); Zhanwu DAI, Beijing (CN); Wei DUAN, Beijing (CN)

(21) Appl. No.: 19/340,042

(22) Filed: Sep. 25, 2025

(30) Foreign Application Priority Data
Oct. 30, 2024 (CN) 202411527689.3

(51) Int. Cl. A01H 1/04; C12Q 1/686; C12Q 1/6895

(52) U.S. Cl. CPC A01H 1/045; C12Q 1/686; C12Q 1/6895; C12Q 2600/13

(57) ABSTRACT

A method for breeding a new grape variety with red flesh and seedlessness based on molecular markers is provided. The molecular markers are identified significantly associated with grape red flesh and seedlessness traits through whole-genome analysis. The molecular markers may identify the phenotypic traits of grape red flesh and seedlessness at the initial stage of hybrid seedlings by using the electrophoretic banding patterns of amplified molecular marker gene fragments.

### CROSS-REFERENCE TO RELATED APPLICATIONS

This application claims priority to Chinese Patent Application No. 202411527689.3, filed on Oct. 30, 2024, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: SequenceListing.xml
Creation date: Sep. 22, 2025
Byte size: 10,489

### TECHNICAL FIELD

The present disclosure relates to the field of biological breeding, and particularly relates to a method for breeding a new grape variety with red flesh and seedlessness based on molecular markers.

### BACKGROUND

China is a major producer and consumer of table grapes. Since 2010, China's grape production has ranked first in the world, with 80% of the produced grapes being table grapes, accounting for approximately half of the world's total table grape production.

Variety is one of the key factors determining fruit quality, and adopting superior varieties is a prerequisite for producing high-quality fruits. Currently, 70% of the grape varieties used in China's production are introduced from abroad. These varieties exhibit poor adaptability to China's climatic conditions, creating an urgent need for the breeding and promotion of high-quality grape varieties with independent intellectual property rights.

Convenient-to-cat seedless grapes have always been favored by consumers and remain a key breeding target of breeders. In the U.S., seedless grapes already account for 90% of table grape production. Chile has also seen rapid development in seedless grape cultivation, with its varietal structure gradually shifting from seeded to seedless types-currently exceeding 50% in cultivation area. It may be said that seedless grapes represent an inevitable trend in the table grape industry. Currently, China's breeding of new seedless grape varieties still relies mainly on conventional hybridization. Although embryo rescue technology may improve the probability of obtaining seedless progeny in hybrid populations, the final selection still requires waiting until the hybrid seedlings are planted and stably bear fruit, a process that takes at least five years, resulting in low efficiency and high costs. While some seedless molecular markers have been reported, most studies focus on marker development and validation. There have been no reports on the practical application of a single seedless-related marker for progeny screening and variety selection.

Fruit quality and nutritional value are also critical factors determining the economic worth of varieties. Anthocyanins in berries not only directly influence consumer preference but also provide significant health benefits. Breeding new seedless grape varieties with high quality is an urgent need for China's grape industry and a key objective in functional variety development. Therefore, there is a pressing demand to accelerate the breeding of high-quality (high anthocyanin content) seedless grape varieties with independent intellectual property rights. However, no molecular markers for red flesh have been reported to date, nor have molecular markers combining seedlessness with high-quality traits been applied in practical breeding. Using genomic technologies for molecular marker-assisted breeding targeting multiple traits is an inevitable trend in China's grape germplasm innovation and a necessary means to enhance germplasm innovation capabilities.

### SUMMARY

The objective of the present disclosure is to provide a method for breeding a new grape variety with red flesh and seedlessness based on molecular markers, addressing the aforementioned problems in existing technologies. By mining genome-wide data, broad-spectrum molecular markers significantly associated with red flesh and seedlessness traits are obtained, and a method for breeding a new grape variety with red flesh and seedlessness is established. This method may accurately and efficiently screen a new grape variety with red flesh and seedlessness that meets the breeding objectives at the initial stage of hybrid seedlings, thereby improving breeding efficiency and reducing costs.

To achieve the above objective, the present disclosure provides the following schemes:

the disclosure provides a molecular marker for identifying a new grape variety with red flesh and seedlessness, where the molecular marker consists of a molecular marker R1 for identifying the red flesh in grape berries and a molecular marker S1 for identifying the seedlessness in grapes, where the molecular marker R1 is located in an upstream promoter region of a key grape gene VvMYBA1, the molecular marker R1 contains ≥2 copies of MYBA1 promoter, where a promoter sequence is as shown in SEQ ID NO: 2, a nucleotide sequence of the molecular marker S1 is as shown in SEQ ID NO: 3, and a 70-base-pair (bp) deletion starting from position 121 is present in the sequence, and a deleted fragment is as shown in SEQ ID NO: 4.

The disclosure further provides a method for breeding the new grape variety with the red flesh and the seedlessness, including steps of using the molecular marker to breed the new grape variety with the red flesh and the seedlessness.

Optionally, the method includes the following steps:

extracting DNA from young tissues of grape hybrid seedlings, amplifying the molecular marker S1 using a primer pair as shown in SEQ ID NO: 5-6, amplifying the molecular marker R1 using a primer pair as shown in SEQ ID NO: 7-8, and breeding the new grape variety with the red flesh and the seedlessness based on a banding pattern of amplification products.

Optionally, parents of the hybrid seedlings include at least one red-fleshed variety parent and one seedless variety parent.

Optionally, an amplification reaction system includes: 25 microliters (μL) of 2×Rapid Taq Master Mix, 2 μL of upstream primer, 2 μL of downstream primer, 0.1-1 microgram (μg) of DNA template, with ddH₂O added to reach total volume of 50 μL.

Optionally, an amplification reaction program is: 94 degrees Celsius (° C.) denaturation for 5 minutes (min), 94° C. denaturation for 30 seconds(s), 59° C. annealing for 30 s with annealing temperature decreasing by 0.5° C. per cycle, 72° C. extension for 30 s, for a total of 5 cycles; 94° C. denaturation for 30 s, 56° C. annealing for 30 s, 72° C. extension for 30 s, for a total of 25 cycles; and followed by a final extension at 72° C. for 5 min, and storage at 4° C.

Optionally, the molecular marker R1 exhibits a double-band of 638 bp and (638+408n) bp for red-fleshed grapes and a single band of 638 bp for white-fleshed grapes, where n is a natural number≥1, and the molecular marker S1 exhibits a single band of 284 bp for seeded grapes and a band of 214 bp for seedless grapes.

Optionally, a hybrid seedling variety retaining the double-band of 638 bp and (638+408n) bp for the molecular marker R1 and the band of 214 bp for the molecular marker S1 is a new hybrid grape variety with the red flesh and the seedlessness.

The present disclosure further provides a primer pair for identifying new varieties with red flesh and seedlessness, where the nucleotide sequences of the primer pair are as shown in SEQ ID NO: 5-8, with the primer pair of SEQ ID NO: 5-6 being used to amplify the molecular marker S1, and the primer pair of SEQ ID NO: 7-8 being used to amplify the molecular marker R1.

The disclosure further provides an application of the molecular markers or the primer pairs in breeding new grape varieties.

The disclosure has the following technical effects.

Through whole-genome analysis, the disclosure has identified molecular markers (namely molecular markers R1 and S1) significantly associated with grape red flesh and seedlessness traits. Based on these molecular markers, a method suitable for breeding new grape varieties with both red flesh and seedlessness has been developed. This method employs agarose gel electrophoresis detection, which is rapid, convenient, produces stable results unaffected by environmental factors, and enables accurate screening of grape individuals with red flesh and seedlessness traits. Identification and selection of hybrid progeny may be conducted at the seedling stage, thereby reducing the breeding cycle, saving substantial land and management inputs, and improving breeding efficiency.

Compared with conventional breeding methods in prior art, the disclosure provides broad-spectrum single seedless markers combined with red flesh markers, enabling simultaneous early-stage identification and selection of seedless and red-fleshed traits in hybrid progeny. This approach reduces the breeding cycle by at least 5 years compared to conventional breeding methods. The molecular markers and breeding method provided by the disclosure may be used for early screening of hybrid grapes with red flesh and seedlessness traits, providing support for efficient molecular-assisted breeding of new grape varieties with red flesh and seedlessness.

### BRIEF DESCRIPTION OF THE DRAWINGS

To more clearly illustrate the technical schemes in the embodiments of the present disclosure or the prior art, the following briefly describes the accompanying drawings required for the embodiments. Obviously, the drawings in the following description show only some embodiments of the present disclosure. For those of ordinary skill in the art, other drawings may be derived from these illustrations without creative effort.

FIG. 1 shows the banding pattern for identifying red-fleshed progeny lines in the population (red-fleshed lines exhibit double bands); where from left to right, the bands represent: Marker, ‘No. 19’ (red flesh), ‘No. 35’ (red flesh), ‘No. 38’ (red flesh), ‘No. 69’ (red flesh), ‘No. 82’ (red flesh), ‘No. 85’ (red flesh), and ‘No. 136’ (white flesh).

FIG. 2 shows the banding pattern for identifying seedless progeny lines in the population (seedless lines show bands at a distinct position); where from left to right, the bands represent: ‘No. 19’ (seeded), ‘No. 35’ (seedless), ‘No. 38’ (seedless), ‘No. 69’ (seedless), ‘No. 82’ (seeded), ‘No. 85’ (seedless), ‘No. 136’ (seeded), and Marker.

FIG. 3 is a flow chart of a method for breeding a new grape variety with red flesh and seedlessness.

### DETAILED DESCRIPTION OF THE EMBODIMENTS

A number of exemplary embodiments of the present disclosure will now be described in detail, and this detailed description should not be considered as a limitation of the present disclosure, but should be understood as a more detailed description of certain aspects, characteristics and embodiments of the present disclosure.

It should be understood that the terminology used in the present disclosure is only for describing specific embodiments and is not used for limiting the present disclosure. In addition, for the numerical range in the present disclosure, it should be understood that each intermediate value between the upper limit and the lower limit of the range is also specifically disclosed. Intermediate values within any stated value or stated range, as well as each smaller range between any other stated value or intermediate values within the stated range are also included in the present disclosure. The upper and lower limits of these smaller ranges may be included or excluded independently.

Unless otherwise specified, all technical and scientific terms used herein have the same meaning as commonly understood by one of ordinary skill in the art to which this disclosure relates. Although the present disclosure only describes the optional methods and materials, any methods and materials similar or equivalent to those described herein may also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the present specification without departing from the scope or spirit of the present disclosure. Other implementation modes obtained from the description of the present disclosure are obvious to the skilled person. The description and embodiments of the present disclosure are exemplary only.

The terms “comprising”, “including”, “having” and “containing” used herein are all open terms, which means including but not limited to.

### Embodiment 1 A Molecular Marker for Identifying a New Grape Variety with Red Flesh and Seedlessness

The present disclosure obtains whole-genome sequencing data from the red-fleshed grape variety ‘Yan 73’ and the seedless grape variety ‘Seedless White’, and conducts comparative genomic analysis with Vitis vinifera varieties ‘Cabernet Sauvignon’, ‘Pinot Noir’, ‘Chardonnay’ and wild Vitis vinifera species. Through integration of gene expression and linkage analysis, broad-spectrum molecular markers for red flesh and seedlessness traits are identified:

characteristics of the red flesh molecular marker (REDFLESH, designated R1): this molecular marker is located in the upstream promoter region of the key anthocyanin regulatory gene VvMYBA1, with the nucleotide sequence shown in SEQ ID NO:1. The molecular marker represents a copy number variation (CNV) molecular marker tightly linked to flesh color traits. The copy number of promoter variation of the molecular marker affects VvMYBA1 expression. When the promoter copy number is ≥2, VvMYBA1 is expressed in the flesh, resulting in red flesh coloration. At the same time, a naturally occurring single-copy segment in the grape genome serves as a control, making this marker suitable for flesh color identification.

Characteristics of the seedlessness molecular marker (SEEDLESS, designated S1): Genome-wide association study (GWAS) is employed to conduct whole-genome analysis of the seedlessness trait, identifying multiple single nucleotide polymorphism (SNP) loci in the MADS8 gene. Through linkage analysis and comprehensive investigation of resequencing data, a 70 base pair (bp) insertion-deletion (InDel) exhibiting high linkage with these SNPs is discovered. The sequence of this InDel molecular marker is presented in SEQ ID NO: 3, which contains a 70 bp deletion starting at position 121 (the deleted sequence is shown in SEQ ID NO: 4). Validation confirms that this InDel shows strong correlation with grape seedlessness traits, thus serving as an effective marker for distinguishing between seeded and seedless varieties.

The sequence of SEQ ID NO: 1 is as follows:

CAGTGAGGGTAACAAAGTCAATTTTTTTAACATTGGGTATATGTATATATT
TGAGGGAATTGTCAGTGAGAAGAATACATGGTAGGAAATGACACACGTCCCAAGCA
ACAGATGGATGGTTGAGAATGAACCCGGTCATTGAATTGACATTAGAAAATGTTCT
AAAATATATTTTTATCGACGTTACTGATCAGAAACAAAGTCCTCCGCCAGCCAGAGG
CATATCCTAATGATTGTACCTTTCCTCACTGACAATTCCCATTAATTAGCTGCTGCCA
CTGCATAGCGGCTATAATATAATGGTAGAGGGCCCATGGAGCTTTCCTTTTCAGTGA
ACATGGGTTAGTCGACAAAAGAAAATGTTCAAGTTGAAAGAGGAGCGGTGGCCCTC
AAAGGTTCCCGTCACTTGGTTGCTTTTTGTCTAAGGAAACAGTGGTATCAGAATCCA

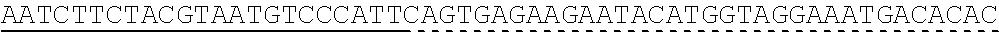

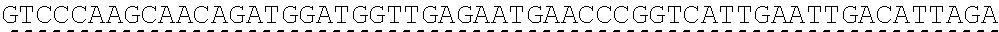

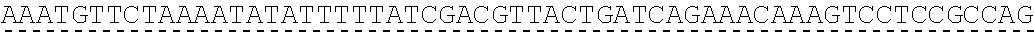

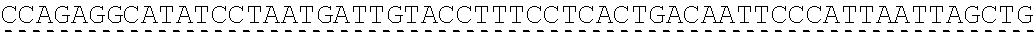

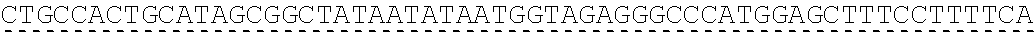

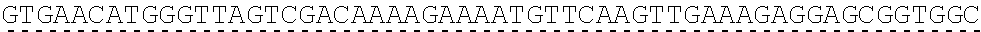

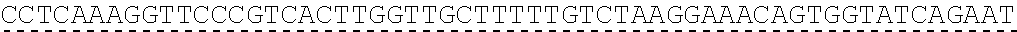

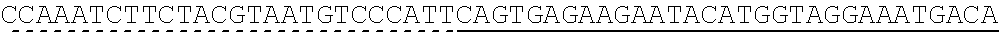

CACGTCCCAAGCAACAGATGGATGGTTGAGAATGAACCCGGTCATTGAATTGACAT
TAGAAAATGTTCTAAAATATATTTTTATCGACGTTACTGATCAGAAACAAAGTCCTC
CGCCAGCCAGAGGCATATCCTAATGATTGTACCTTTCCTCACTGACAATTCCCATTA
ATTAGCTGCTGCCACTGCATAGCGGCTATAATATAATGGTAGAGGGCCCATGGAGCT
TTCCTTTTCAGTGAACATGGGTTAGTCGACAAAAGAAAATGTTCAAGTTGAAAGAG
GAGCGGTGGCCCTCAAAGGTTCCCGTCACTTGGTTGCTTTTTGTCTAAGGAAACAGT
GGTATCAGAATCCAAATCTTCTACGTAATGTCCCATTCATCCTACCAATGTCCATAT
GAATTCCTCTGGACGTTAAAAAATGGTTGCACGTGGTTGTCTTCAGGATCACACCAG
TTTATACATTTGGACCACAAAATAGAGATTGTTCATCAAGGATACTAGTCAGCAATT
AATTCCTAAATATCTCTTATGACACACACCCTT.

The underlined part of the sequence is the 408 bp sequence (SEQ ID NO: 2) with 408×3 bp of MYBA1 promoter variation.

The sequence of SEQ ID NO: 3 is as follows:

TTCAGGATAGGGAGGTCTGAACTCAGTTGAAGGAATAGTAAAAGAAGAC
AAAGCAACTATTAGAGCTTAAGAAGAGTCCCATTTTAGTATTAGTGACT
TCATACTACAAAGAAAACTTAGGGAAAAAAAAAACTCAATTAGAAAAAA
GTCTTTTTGACAAGATTAGGATTACTGATATGGGTCTTAGCATGTAAGC
AATCATGTTCATGCATTCCAATTCTTTCTATTCTTGTTTTCCTTTTTTC
CTTTTTTTCACCTAATATTTGTTGAGATGAAAAGTGGCT.

The underlined part represents the deleted 70 bp sequence (SEQ ID NO: 4).

### Embodiment 2 A Method for Breeding a New Grape Variety with Red Flesh and Seedlessness

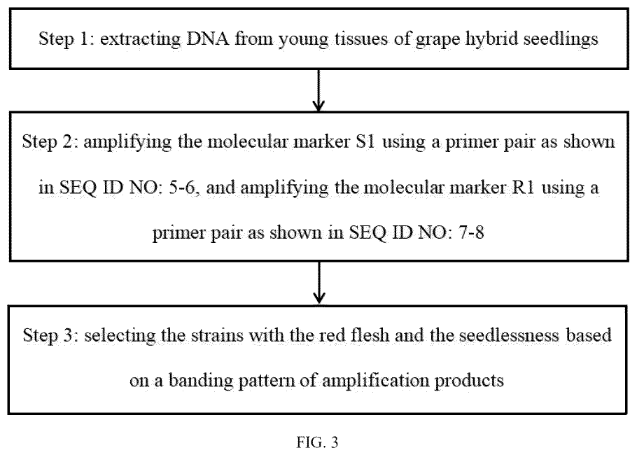

The disclosure provides a method for breeding the new grape variety with the red flesh and the seedlessness, the steps are shown in FIG. 3, where the specific steps are as follows.

### 1. Hybrid Combination Configuration

Suitable parents are selected to establish hybrid combinations. When breeding new superior varieties with seedlessness and red flesh characteristics, the hybrid combination must include one red-fleshed variety parent and one seedless variety parent. The red-fleshed variety parent may serve as either the female or male parent. If the seedless variety parent is used as the female parent, embryo culture is required after flowering to obtain hybrid seedlings through embryo rescue. The flowering period of the male parent variety must coincide with or precede the flowering period of the female parent.

### 2. Hybrid Seedling Production

When seeded varieties serve as female parents, mature hybrid seeds are obtained through crossing and subjected to alternating 20/30 degrees Celsius (° C.) temperature treatment to promote germination of hybrid seedlings. When seedless varieties serve as female parents, hybrid seedlings are obtained through embryo rescue.

### 3. Sampling and DNA Extraction

Young tissues such as leaves and stems are collected from hybrid seedlings. DNA is extracted using a modified cetyltrimethylammonium bromide (CTAB) plant genomic DNA rapid extraction kit. The extracted DNA is diluted to approximately 200 nanograms per microliter (ng/μL) and stored at −20° C.

### 4. Polymerase Chain Reaction (PCR) Amplification

### 4.1 PCR Detection Primers

Primers are designed based on the seedless molecular marker S1 and red flesh molecular marker R1 provided in this disclosure:

Seedless molecular marker S1:
F (SEQ ID NO: 5): 5′-TTCAGGATAGGGAGGTCTGAA-3′;
R (SEQ ID NO: 6): 5′-AGCCACTTTTCATCTCAACAAA-3′.
Red flesh molecular marker R1:
F (SEQ ID NO: 7): 5′-CAGTGAGGGTAACAAAGTCA-3′;
R (SEQ ID NO: 8): 5′-AAGGGTGTGTGTCATAAGAG-3′.

Detection is performed using these primers.

### 4.2 PCR Reaction System (50 Microliters (μL))

All operations are performed on ice. The total PCR amplification reaction system is 50 μL as follows:

TABLE 1
Reaction system

| Reagent | Dosage |
|---|---|
| 2 × Rapid Taq Master Mix | 25 μL |
| Upstream primer F (10 micromolar (μM)) | 2 μL |
| Downstream primer R (10 μM) | 2 μL |
| Template DNA | 0.1-1 microgram (μg) |
| ddH₂O | Making up to a total |
| | volume of 50 μL |

### 4.3 PCR Amplification Program

To improve the specificity of amplification products, target fragments are amplified using Touchdown-PCR with the following program: 94° C. denaturation for 5 minutes (min), 94° C. denaturation for 30 seconds(s), 59° C. annealing for 30 s with annealing temperature decreasing by 0.5° C. per cycle, 72° C. extension for 30 s, for a total of 5 cycles; 94° C. denaturation for 30 s, 56° C. annealing for 30 s, 72° C. extension for 30 s, for a total of 25 cycles; and followed by a final extension at 72° C. for 5 min, and storage at 4° C.

### 4.4 Agarose Gel Electrophoresis

1.5 grams (g) agarose powder is added to 100 milliliters (mL) 1×Tris-Acetate-EDTA (TAE) buffer (1.5% agarose gel) in a conical flask. After mixing, the solution is heated in a microwave until completely dissolved. 10 μL nucleic acid dye is added to the transparent solution. The solution is poured into a gel tray with a comb inserted. After solidification, the comb is removed and the gel is placed in an electrophoresis tank. 5-10 μL PCR product is loaded into each well. Electrophoresis is performed at constant 140 volts (V) for 15-30 min. After electrophoresis, results are observed and photographed under an ultraviolet gel imaging system.

### 4.5 Result Verification

The amplified product sequence of the red flesh marker is shown in SEQ ID NO: 1, while the amplified product sequence of the seedless marker is shown in SEQ ID NO: 3. Clear and uniform electrophoretic bands indicate proper DNA purity and concentration. For the red flesh marker, two bands (638 bp and (638+408n) bp, where n≥1) indicate red-fleshed varieties, while a single 638 bp band indicates white-fleshed varieties. The seedless marker shows a 70 bp deletion, causing seedless bands to migrate farther than seeded bands, appearing lower on the agarose gel relative to the seeded grape band. By comparing the banding patterns, the seedlessness status of the variety may be precisely determined.

### Embodiment 3

The method for breeding new grape varieties described in Embodiment 2 is applied and validated using the hybrid progeny population derived from the cross between ‘Yan 73’ (red-fleshed variety)×‘Zixiang Seedless’ (seedless variety) as an example.

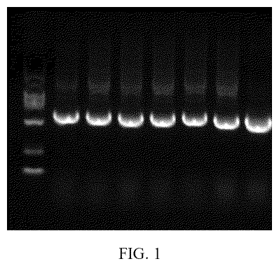

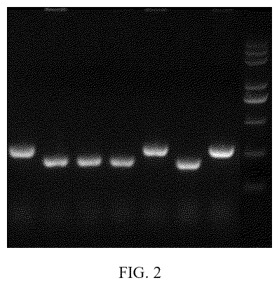

Following the method described in Embodiment 2, four grape lines (′No. 35′, ‘No. 38’, ‘No. 69’, and ‘No. 85’) with both red flesh and seedlessness traits that meet the breeding objectives are selected from the hybrid progeny population of ‘Yan 73’ (red-fleshed variety)×‘Zixiang Seedless’ (seedless variety). The electrophoretic results are shown in FIG. 1 and FIG. 2. The results demonstrate that all red-fleshed grapes display double bands (638 bp, and 638 bp and (638+408n) bp), while seedless grapes exhibit bands at more distant positions compared to seeded grapes. Seeded grapes show a single band at 284 bp whereas seedless grapes show a single band at 214 bp. Among these, ‘No. 69’ demonstrates superior comprehensive characteristics and is selected as a breeding line, which receives plant variety rights application acceptance in 2022 and currently enters the substantive examination phase.

At present, using seeded varieties as female parents and seedless varieties as male parents, the lowest proportion of seedless lines in hybrid progeny is only 10%-15%, and most of them are seeded lines (Reynolds, 2014). Even when seedless female parents are crossed with seedless male parents, while the probability of seedless lines in the hybrid progeny increases substantially, at least 25% of the hybrid progeny remain seeded lines. Even seedless lines, their seed abortion may not meet the breeding objective of seedless new varieties (Ramming, 1990). In addition, conventional screening methods require planting all hybrids in fields and waiting through juvenile periods until fruit set to determine whether the lines are seedless, and the process will take at least 5-6 years. Therefore, if early screening of hybrid progeny using seedless markers is implemented, firstly, the breeding process is accelerated with at least a 5-year reduction in the breeding cycle; secondly, substantial land occupation and management resources required for transplanted hybrid seedlings are reduced, thereby decreasing breeding costs. The combination of seedlessness markers with other quality markers further highlights the advantages of modern biotechnological breeding and enhances breeding efficiency. The results from the aforementioned embodiments demonstrate that the molecular markers provided by the present disclosure, along with the breeding method developed based on the molecular markers, enable determination of seedlessness and flesh color traits in progeny by the second year after hybridization, allowing targeted selection of desired trait combinations and significantly shortening the breeding duration. The technical schemes proposed in the present disclosure represent an important discovery and advancement in grape breeding, possessing substantial practical significance and value.

The above-mentioned embodiments only describe the optional mode of the disclosure, and do not limit the scope of the disclosure. Under the premise of not departing from the design spirit of the disclosure, various modifications and improvements made by ordinary technicians in the field to the technical schemes of the disclosure shall fall within the protection scope determined by the claims of the disclosure.

What is claimed is:

1. A molecular marker for identifying a new grape variety with red flesh and seedlessness, wherein the molecular marker comprises a molecular marker R1 for identifying the red flesh in grape berries and a molecular marker S1 for identifying the seedlessness in grapes, wherein the molecular marker R1 is located in an upstream promoter region of a key grape gene VvMYBA1, the molecular marker R1 comprises ≥2 copies of MYBA1 promoter, wherein a promoter sequence is as shown in SEQ ID NO: 2, a nucleotide sequence of the molecular marker S1 is as shown in SEQ ID NO: 3, and a 70-base-pair (bp) deletion starting from position 121 is present in the sequence, and a deleted fragment is as shown in SEQ ID NO: 4.

2. A method for breeding the new grape variety with the red flesh and the seedlessness, comprising steps of using the molecular marker according to claim 1 to breed the new grape variety with the red flesh and the seedlessness.

3. The method according to claim 2, comprising following steps: extracting deoxyribonucleic acid (DNA) from young tissues of grape hybrid seedlings, amplifying the molecular marker S1 using a first primer pair as shown in SEQ ID NO: 5-6, amplifying the molecular marker R1 using a second primer pair as shown in SEQ ID NO: 7-8, and breeding the new grape variety with the red flesh and the seedlessness based on a banding pattern of amplification products.

4. The method according to claim 3, wherein parents of the grape hybrid seedlings comprise at least one red-fleshed variety parent and one seedless variety parent.

5. The method according to claim 3, wherein an amplification reaction system comprises: 25 microliters (μL) of 2×Rapid Taq Master Mix, 2 μL of upstream primer, 2 μL of downstream primer, 0.1-1 microgram (μg) of DNA template, with ddH₂O added to reach total volume of 50 μL.

6. The method according to claim 3, wherein an amplification reaction program is: 94 degrees Celsius (° C.) denaturation for 5 minutes (min), 94° C. denaturation for 30 seconds(s), 59° C. annealing for 30 s with annealing temperature decreasing by 0.5° C. per cycle, 72° C. extension for 30 s, for a total of 5 cycles; 94° C. denaturation for 30 s, 56° C. annealing for 30 s, 72° C. extension for 30 s, for a total of 25 cycles; and followed by a final extension at 72° C. for 5 min, and storage at 4° C.

7. The method according to claim 3, wherein the molecular marker R1 exhibits a double-band of 638 bp and (638+408n) bp for red-fleshed grapes and a single band of 638 bp for white-fleshed grapes, wherein n is a natural number≥1, and the molecular marker S1 exhibits a single band of 284 bp for seeded grapes and a band of 214 bp for seedless grapes.

8. The method according to claim 7, wherein a hybrid seedling variety retaining the double-band of 638 bp and (638+408n) bp for the molecular marker R1 and the band of 214 bp for the molecular marker S1 is a new hybrid grape variety with the red flesh and the seedlessness.
